(19) United States
(12) Patent Application Publication
(10) Pub. No.: US 2024/0428952 A1
(43) Pub. Date: Dec. 26, 2024

(54) DEVICE AND METHOD FOR PREDICTING PATHOLOGIC FRACTURE IN PROXIMAL FEMUR, AND NON-TRANSITORY COMPUTER-READABLE STORAGE MEDIUM STORING PROGRAM FOR PERFORMING THE METHOD

(71) Applicant: The Catholic University of Korea Industry-Academic Cooperation Foundation, Seoul (KR)

(72) Inventor: Min Wook JOO, Suwon (KR)

(21) Appl. No.: 18/750,890

(22) Filed: Jun. 21, 2024

(30) Foreign Application Priority Data
Jun. 23, 2023 (KR) 10-2023-0081146
Dec. 19, 2023 (KR) 10-2023-0186041

(51) Int. Cl. G16H 50/50; G06T 7/00; G16H 30/40

(52) U.S. Cl. CPC G16H 50/50; G06T 7/0012; G16H 30/40; G06T 2207/10081; G06T 2207/10124; G06T 2207/20081; G06T 2207/20084; G06T 2207/30008

(57) ABSTRACT

A device and method for predicting a pathologic fracture in the proximal femur are disclosed. The device for predicting a pathologic fracture in the proximal femur according to an aspect of the present invention is a device for predicting a pathologic fracture in the proximal femur of an advanced cancer patient, including: a memory for storing one or more instructions; and a processor for executing the one or more instructions, wherein the processor can process a CT image including the femur of the cancer patient to generate an input image by executing the one or more instructions, input the input image into a fracture prediction artificial neural network model, and obtain prediction information about whether a pathologic fracture in the proximal femur will occur within a predetermined period from the time of acquiring the CT image which is output by the fracture prediction artificial neural network model.

### CROSS-REFERENCE TO RELATED APPLICATIONS

This application claims benefit of priority to Korean Patent Application No. 10-2023-0081146 filed on Jun. 23, 2023, and Korean Patent Application No. 10-2023-0186041 filed on Dec. 19, 2023, the disclosures of which are incorporated herein by reference in their entirety.

### FIELD OF THE INVENTION

The present invention relates to a device and method for predicting a pathologic fracture in the proximal femur, and a non-transitory computer-readable storage medium storing a program for performing the method, and more specifically to a device and method for predicting a pathologic fracture in the proximal femur that predict the possibility of development of a pathologic fracture in the proximal femur from medical imaging data of patients with advanced cancer, and a non-transitory computer-readable storage medium storing a program for performing the method.

### BACKGROUND

Improvement in the survival of patients with advanced cancer is accompanied by an increased probability of bone metastasis and related pathologic fractures (especially in the proximal femur). If cancer spreads to the bone and a pathologic fracture develops, treatment and recovery are very difficult, and thus, it is effective to predict the same in advance and take related measures before a fracture occurs.

However, in the past, no highly reliable technology has been developed to predict pathologic fractures in patients with advanced cancer, and generally, there is no choice but to rely on the clinical judgment of medical doctors. In this situation, there is a need for the development of new screening technologies that can diagnose impending fractures due to cancer metastasis and ultimately prevent future fractures.

The present invention has been devised to solve the above problems, and an object of the present invention is to provide a device and method for predicting a pathologic fracture in the proximal femur that can efficiently predict the possibility of development of a pathologic fracture in the proximal femur of patients with advanced cancer, and a non-transitory computer-readable storage medium storing a program for performing the method.

Another object of the present invention is to provide a device and method for predicting a pathologic fracture in the proximal femur that can predict the possibility of development of a pathologic fracture in the proximal femur of patients with advanced cancer without additional test procedures and data by utilizing abdomen-pelvic CT scans that are regularly taken as a standard modality for staging and follow-up of cancer patients, and a non-transitory computer-readable storage medium storing a program for performing the method.

The problems of the present invention are not limited to the problems mentioned above, and other problems that are not mentioned will be clearly understood by those skilled in the art from the description below.

According to an aspect of the present invention, provided is a device for predicting a pathologic fracture in the proximal femur of an advanced cancer patient, including a memory for storing one or more instructions; and a processor for executing the one or more instructions, wherein the processor processes a CT image including the femur of the cancer patient to generate an input image by executing the one or more instructions, inputs the input image into a fracture prediction artificial neural network model, and obtains prediction information about whether a pathologic fracture in the proximal femur will occur within a predetermined period from the time of acquiring the CT image which is output by the fracture prediction artificial neural network model.

In the device for predicting a pathologic fracture in the proximal femur according to an aspect of the present invention, the CT image may be a CT image of the abdomen and pelvis of the cancer patient.

In the device for predicting a pathologic fracture in the proximal femur according to an aspect of the present invention, the processor may reconstruct the CT image into a radiograph image to generate the input image.

In the device for predicting a pathologic fracture in the proximal femur according to an aspect of the present invention, the CT image may be a three-dimensional image and the radiograph image may be a two-dimensional image, and wherein the processor converts the CT image into the radiograph image through perspective projection.

In the device for predicting a pathologic fracture in the proximal femur according to an aspect of the present invention, the processor may generate a cropped radiograph image by extracting a part representing the femur of the cancer patient from the reconstructed radiograph image.

In the device for predicting a pathologic fracture in the proximal femur according to an aspect of the present invention, the processor may augment the cropped radiograph image to generate a plurality of input images from one of the cropped radiograph images.

In the device for predicting a pathologic fracture in the proximal femur according to an aspect of the present invention, the fracture prediction artificial neural network model may be trained by receiving inputs of a learning input image generated by processing abdomen and pelvis CT images of a plurality of cancer patients, and the determination of whether a pathologic fracture in the proximal femur will occur in each of the plurality of cancer patients within a predetermined period from the time of acquiring the CT images, and wherein the learning input image is generated by respectively reconstructing the abdomen and pelvis CT images of the plurality of cancer patients into radiograph images, and extracting a part representing the femur of each of the cancer patients from each of the reconstructed radiograph images such that a generated femur cropped image is augmented.

According to another aspect of the present invention, provided is a method for predicting a pathologic fracture in the proximal femur of an advanced cancer patient, including the steps of receiving an input of a CT image including the femur of the cancer patient; generating an input image by processing the CT image; and inputting the input image into a fracture prediction artificial neural network model, and obtaining prediction information about whether a pathologic fracture in the proximal femur will occur within a predetermined period from the time of acquiring the CT image which is output by the fracture prediction artificial neural network model.

In the method for predicting a pathologic fracture in the proximal femur according to another aspect of the present invention, the CT image may be a three-dimensional CT image of the abdomen and pelvis of the cancer patient.

In the method for predicting a pathologic fracture in the proximal femur according to another aspect of the present invention, the step of generating an input image may include the steps of reconstructing the three-dimensional CT image into a two-dimensional radiograph image; generating a cropped radiograph image by extracting a part representing the femur of the cancer patient from the reconstructed radiological image; and augmenting the cropped radiograph image to generate a plurality of input images from one of the cropped radiograph images.

In the method for predicting a pathologic fracture in the proximal femur according to another aspect of the present invention, the three-dimensional CT image may be reconstructed into the two-dimensional radiograph image through perspective projection.

In the method for predicting a pathologic fracture in the proximal femur according to another aspect of the present invention, the fracture prediction artificial neural network model may be trained by receiving inputs of a learning input image generated by processing abdomen and pelvis CT images of a plurality of cancer patients, and the determination of whether a pathologic fracture in the proximal femur will occur in each of the plurality of cancer patients within a predetermined period from the time of acquiring the CT images, and wherein the learning input image is generated by respectively reconstructing the abdomen and pelvis CT images of the plurality of cancer patients into radiograph images, and extracting a part representing the femur of each of the cancer patients from each of the reconstructed radiograph images such that a generated femur cropped image is augmented.

According to still another aspect of the present invention, provided is a non-transitory computer-readable storage medium storing a program including one or more instructions for performing the method for predicting a pathologic fracture in the proximal femur according to another aspect of the present invention.

According to the above configurations, the device and method for predicting a pathologic fracture in the proximal femur according to an exemplary embodiment of the present invention and a non-transitory computer-readable storage medium storing a program for performing the method can efficiently predict the possibility of development of a pathologic fracture in the proximal femur of patients with advanced cancer through a trained artificial neural network model.

The device and method for predicting a pathologic fracture in the proximal femur according to an exemplary embodiment of the present invention and a non-transitory computer-readable storage medium storing a program for performing the method can predict the possibility of development of a pathologic fracture in the proximal femur of patients with advanced cancer without additional test procedures and data by utilizing abdomen-pelvic CT scans that are regularly taken as a standard modality for staging and follow-up of cancer patients.

The effects of the present invention are not limited to the effects described above, and it should be understood to include all effects that can be inferred from the configuration of the invention described in the detailed description or claims of the present invention.

### BRIEF DESCRIPTION OF THE DRAWINGS

FIG. 1 is a diagram showing the configuration of the device for predicting a pathologic fracture in the proximal femur according to an exemplary embodiment of the present invention.

FIG. 2 is a diagram exemplarily showing the generation process of an input image which is input into an artificial neural network model in the device for predicting a pathologic fracture in the proximal femur according to an exemplary embodiment of the present invention.

FIG. 3 is a diagram showing an exemplary configuration of an artificial neural network model used in the device for predicting a pathologic fracture in the proximal femur according to an exemplary embodiment of the present invention.

FIG. 4 is a flowchart of the method for predicting a pathologic fracture in the proximal femur according to an exemplary embodiment of the present invention.

FIG. 5 is a detailed flowchart of the step of generating an input image in the method for predicting a pathologic fracture in the proximal femur according to an exemplary embodiment of the present invention.

FIG. 6 shows a receiver operating characteristic (ROC) curve when the artificial neural network model used in the device and method for predicting a pathologic fracture in the proximal femur according to an exemplary embodiment of the present invention is configured by DenseNet121, and the input image generated from the CT image is used.

### DETAILED DESCRIPTION

Hereinafter, with reference to the attached drawings, the exemplary embodiments of the present invention will be described in detail so that those skilled in the art can easily practice the present invention. The present invention may be implemented in many different forms and is not limited to the exemplary embodiments described herein. In order to clearly describe the present invention, parts that are not relevant to the description have been omitted in the drawings, and the same or similar components are assigned the same reference numerals throughout the specification.

The words and terms used in the present specification and claims are not to be construed as limited in their usual or dictionary meanings, but according to the principle that the inventor can define terms and concepts in order to explain his or her invention in the best way, they must be interpreted with meanings and concepts that are consistent with the technical ideas of the present invention.

In the present specification, terms such as “include” or “have” are intended to describe the existence of features, numbers, steps, operations, components, parts or combinations thereof described in the specification, but it should be understood that the terms do not exclude the presence or possibility of addition of one or more other features, numbers, steps, operations, components, parts or combinations thereof.

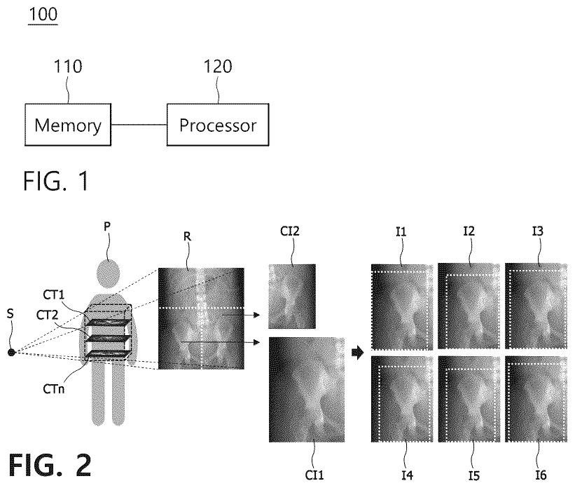

FIG. 1 is a diagram showing the configuration of the device for predicting a pathologic fracture in the proximal femur according to an exemplary embodiment of the present invention.

The device for predicting a pathologic fracture in the proximal femur 100 according to an exemplary embodiment of the present invention predicts the possibility of development of a fracture in the proximal femur of an advanced cancer patient through a trained artificial intelligence model. More specifically, the device for predicting a pathologic fracture in the proximal femur 100 according to an exemplary embodiment of the present invention may predict whether a pathologic fracture will occur in the proximal femur of an advanced cancer patient within a predetermined period from a medical image of the patient.

Referring to FIG. 1, the device 100 for predicting a pathologic fracture in the proximal femur according to an exemplary embodiment of the present invention includes a memory 110 and a processor 120.

The memory 110 stores one or more instructions. The one or more instructions may be performed by the processor 120. More specifically, the one or more instructions enable processor 120 to perform operations to predict whether a pathologic fracture will occur within a predetermined period in the proximal femur of an advanced cancer patient.

The memory 110 may include a semiconductor device-based storage medium such as RAM, ROM, flash memory or the like. In addition, the memory 110 may include magnetic media such as a hard disk, floppy disk, magnetic tape and the like, optical recording media such as a CD-ROM (Compact Disk Read Only Memory), a DVD (Digital Video Disk) and the like, and magneto-optical media such as a floptical disk.

The processor 120 executes the one or more instructions. By executing the one or more instructions, the processor 120 may obtain prediction information about whether a pathologic fracture in the proximal femur will occur in a patient with advanced cancer within a predetermined period.

The processor 120 may be a hardware unit that performs calculations and controls within a computer. For example, the processor 120 may include at least one arithmetic logic unit (ALU) and processing register.

By executing the one or more instructions, the processor 120 processes a CT image including the femur of the cancer patient to generate an input image, inputs the input image into a fracture prediction artificial neural network model, and obtains prediction information about whether a pathologic fracture in the proximal femur will occur within a predetermined period from the time of acquiring the CT image which is output by the fracture prediction artificial neural network.

FIG. 2 is a diagram exemplarily showing the generation process of an input image which is input into an artificial neural network model in the device for predicting a pathologic fracture in the proximal femur according to an exemplary embodiment of the present invention.

Referring to FIG. 2, the CT images (CT1, . . . , CTn) may be CT images of the abdomen and pelvis of a cancer patient (P). The abdomen and pelvis CT scan images of cancer patients are regularly taken during the treatment of cancer patients as a standard modality for staging and follow-up of cancer patients.

As such, according to an exemplary embodiment of the present invention, the possibility of development of a fracture in the proximal femur of an advanced cancer patient may be predicted by utilizing an abdomen-pelvic CT scan that is regularly taken as a standard modality for staging and follow-up of cancer patients. Accordingly, since no additional testing procedures or data are required to predict the possibility of proximal femur fractures in patients with advanced cancer, it is possible to reduce costs and improve the efficiency of diagnosis and treatment.

First of all, the processor 120 may reconstruct the CT images (CT1, . . . , CTn) into a radiograph image (R) to generate input images (I1, I2, I3, I4, I5, I6). As shown in FIG. 2, the CT images (CT1, . . . , CTn) are three-dimensional images (3-dimensional volume scan), and the radiograph images are two-dimensional images that are projected on a plane.

The processor 120 may convert the CT images (CT1, . . . , CTn) into a radiograph image (R) through perspective projection. More specifically, the processor 120 may generate a radiograph image (R) by projecting CT images (CT1, . . . , CTn) based on a virtual radiographic source(S) and digitally reconstructing the same.

A two-dimensional image requires fewer deep learning model parameters than a three-dimensional volume. Therefore, when the processor 120 reconstructs the CT images (CT1, . . . , CTn) having a three-dimensional volume into a two-dimensional radiograph image (R), the computational (inference) efficiency of the artificial neural network model may be improved in the future.

Next, the processor 120 may generate cropped radiograph images (CI1, CI2) by extracting a part representing the femur of the cancer patient from the reconstructed radiograph image (R). The region of interest in relation to predicting the probability of development of a pathologic fracture in the proximal femur of the cancer patient is the femur part of the cancer patient. Since the image part outside the region of interest is not relevant to this prediction, cropped radiograph images (CI1, CI2) may be generated to exclude the part outside the region of interest.

For example, after the processor 120 extracts only the bottom 60% region from the reconstructed radiograph image (R), it may cut the image in half along the width center to create two cropped radiograph images (CI1, CI2). That is, a first cropped radiograph image (CI1) and a second cropped radiograph image (CI2) may be extracted from the reconstructed radiograph image (R).

In other words, the reconstructed radiograph image (R) includes a part representing the patient's left femur and a part representing the right femur. Therefore, two cropped radiograph images may be generated from one reconstructed radiograph image (R).

Finally, the processor 120 may augment the cropped radiograph images (CI1, CI2) to generate a plurality of input images from one cropped radiograph image. Through this, the data set which is input into the artificial neural network model that predicts a pathologic fracture in the proximal femur of the cancer patient may be increased.

Referring to FIG. 2, it can be confirmed that the first cropped radiograph image (CI1) is augmented to generate a first input image (I1), a second input image (I2), a third input image (I3), a fourth input image (I4), a first input image (I1) and a sixth input image (I6). The second cropped radiograph image (CI2) is also augmented in this way, and as a result, 6 additional input images may be generated. That is, a total of 12 input images may be generated from the first cropped radiograph image (CI1) and the second cropped radiograph image (CI2).

The processor 120 may augment the cropped radiograph images (CI1, CI2) by applying a cropping technique. Since the proximal femur is in the lower part of each crop radiograph image, the area to be cropped must contact the lowest part of the image. Therefore, the images may be cropped by using randomly generated rectangles that are aligned at the bottom of each cropped radiograph image. For example, the aspect ratio of the rectangle is the same as the original image, and the size of the rectangle may be set to any value between 85% and 95% of the original image.

Certainly, the above augmentation technique is exemplary. The processor 120 may augment the cropped radiograph image by using various augmentation techniques in addition to the exemplary method described above, and generate a plurality of augmented input images. For example, random rotation, random horizontal flipping and the like may be used for data augmentation. Additionally, changes in image brightness, the addition of noise and the like may be used.

The processor 120 inputs the input image generated by processing the CT image into the fracture prediction artificial neural network model. In addition, the processor 120 obtains prediction information about whether a pathologic fracture in the proximal femur will occur within a predetermined period from the time of acquiring the CT image which is output by the fracture prediction artificial neural network model. For example, the predetermined period may be set to within 3 months after the cancer patient takes a CT image.

The fracture prediction artificial neural network model may be trained by receiving inputs of a learning input image generated by processing abdomen and pelvis CT images of multiple cancer patients, and the determination of whether a pathologic fracture in the proximal femur of each of the multiple cancer patients will occur within a predetermined period from the time of acquiring the CT images.

In this case, the learning input image may be generated by respectively reconstructing the abdomen and pelvis CT images of the plurality of cancer patients into radiograph images, and augmenting a femur cropped image generated by extracting a part representing the femur part of each of the cancer patients from each reconstructed radiograph image.

Meanwhile, the artificial neural network model may be a convolution neural network (CNN) model. For example, the artificial neural network model may be any one of DenseNet121, VGG16 and ResNet50.

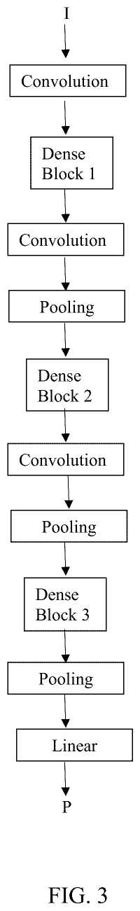

FIG. 3 is a diagram showing an exemplary configuration of an artificial neural network model used in the device for predicting a pathologic fracture in the proximal femur according to an exemplary embodiment of the present invention.

FIG. 3 shows the configuration when the artificial neural network model is DenseNet121. When processing data with a CNN layer, the down-sampling process that changes the size of the feature map is essentially required. DenseNet121 is configured to divide the network into dense blocks and perform CNN and pooling for each dense block.

More specifically, an input image (I) is input, undergoes convolution, undergoes Dense Block 1, convolution and pooling, and then undergoes Dense Block 2, convolution and pooling, and undergoes Dense Block 3, followed by pooling and Linear such that it is possible to output the result (P) of predicting whether a pathologic fracture in the proximal femur will occur within a certain period of time.

The device for predicting a pathologic fracture in the proximal femur according to an exemplary embodiment of the present invention has been reviewed in detail above. Hereinafter, the method for predicting a pathologic fracture in the proximal femur according to an exemplary embodiment of the present invention will be described.

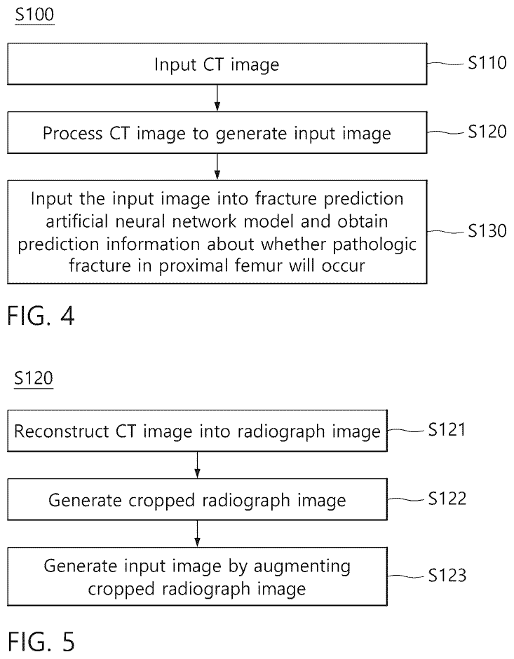

FIG. 4 is a flowchart of the method for predicting a pathologic fracture in the proximal femur according to an exemplary embodiment of the present invention.

The method for predicting a pathologic fracture in the proximal femur (S100) according to an exemplary embodiment of the present invention is a method that is performed on a computer for predicting a pathologic fracture in the proximal femur in patients with advanced cancer. The method for predicting a pathologic fracture in the proximal femur (S100) according to an exemplary embodiment of the present invention may be performed by the device 100 for predicting a proximal femur pathologic fracture according to an exemplary embodiment of the present invention as described above. In addition, the matters described in relation to the device for predicting a pathologic fracture in the proximal femur 100 according to an exemplary embodiment of the present invention may also be applied to the method for predicting a pathologic fracture in the proximal femur (S100) according to an exemplary embodiment of the present invention.

Referring to FIG. 4, the method for predicting a pathologic fracture in the proximal femur (S100) according to an exemplary embodiment of the present invention may be performed as follows. Each of the following steps may be performed by the processor 120 of the device for predicting a pathologic fracture in the proximal femur 100 according to an exemplary embodiment of the present invention by executing one or more instructions stored in the memory 110.

First of all, a CT image including the femur of the cancer patient is input (S110).

The CT image may be a three-dimensional CT image of the abdomen and pelvis of the cancer patient. Abdomen and pelvis CT scan images of cancer patients are regularly taken during the treatment of cancer patients as a standard modality for staging and follow-up of cancer patients.

As such, according to an exemplary embodiment of the present invention, the possibility of development of a fracture in the proximal femur of an advanced cancer patient may be predicted by utilizing an abdomen-pelvic CT scan that is regularly taken as a standard modality for staging and follow-up of cancer patients. Accordingly, costs may be reduced, and the efficiency of diagnosis and treatment may be increased.

Next, the CT image is processed to generate an input image (S120). By processing the CT image to generate an input image, the data set which is input into the artificial neural network model for predicting a pathologic fracture in the proximal femur of the cancer patient may be increased.

Finally, the input image is input into the fracture prediction artificial neural network model, and prediction information regarding whether a pathologic fracture in the proximal femur will occur within a predetermined period from the time of acquiring the CT image output by the fracture prediction artificial neural network model is obtained (S130).

The artificial neural network model may be a convolution neural network (CNN) model. The artificial neural network model may be any one of DenseNet121, VGG16 and ResNet50. For example, the artificial neural network model may be configured by DenseNet121 as illustrated in FIG. 3.

FIG. 5 is a detailed flowchart of the step of generating an input image in the method for predicting a pathologic fracture in the proximal femur according to an exemplary embodiment of the present invention.

Referring to FIG. 5, the step (S120) of processing the CT image to generate an input image in the method (S100) for predicting a pathologic fracture in the proximal femur according to an exemplary embodiment of the present invention may be performed as follows.

First of all, the three-dimensional CT image is reconstructed into a two-dimensional radiograph image (S121). For example, the three-dimensional CT image may be reconstructed into the 2D radiograph image through perspective projection.

Next, a part representing the femur of the cancer patient is extracted from the reconstructed radiograph image to generate a cropped radiograph image (S122). In relation to predicting the possibility of development of a pathologic fracture in the proximal femur of the cancer patient, considering that the region of interest is the femur of the cancer patient, a cropped radiograph image may be generated to exclude image parts outside the region of interest.

Finally, the cropped radiograph image is augmented to generate a plurality of input images from one cropped radiograph image (S123). Through such augmentation of the cropped radiograph image, the data set which is input into the artificial neural network model for predicting a pathologic fracture in the proximal femur of the cancer patient may be increased.

Meanwhile, in the method for predicting a pathologic fracture in the proximal femur (S100) according to an exemplary embodiment of the present invention, the fracture prediction artificial neural network model may be trained by receiving inputs of a learning input image generated by processing abdomen and pelvis CT images of a plurality of cancer patients, and the determination of whether a pathologic fracture in the proximal femur will occur in each of the plurality of cancer patients within a predetermined period from the time of acquiring the CT images

In this case, the learning input image may be generated by respectively reconstructing the abdomen and pelvis CT images of the plurality of cancer patients into radiograph images, and augmenting a femur cropped image generated by extracting a part representing the femur part of each of the cancer patients from each reconstructed radiograph image.

Meanwhile, the present invention additionally provides a non-transitory computer-readable storage medium storing a program for performing the method for predicting a pathologic fracture in the proximal femur. Specifically, the present invention can provide a non-transitory computer-readable storage medium storing a program including one or more instructions for performing the method for predicting a pathologic fracture in the proximal femur.

In this case, the instructions may include not only machine code generated by a compiler but also high-level language code that is executable by a computer. In addition, the storage medium may include hardware devices that are configured to store and execute program instructions, such as magnetic media such as hard disks, floppy disks and magnetic tapes, optical media such as CD-ROM (Compact Disk Read Only Memory) and DVD (Digital Video Disk), magneto-optical media such as a floptical disk, ROM, RAM, flash memory and the like.

In order to verify the effects of the present invention, the fracture prediction artificial neural network model was configured by DenseNet121, and learning input images were generated from 1,971 cases with an average age of 59±12 years (60 cases of pathologic fractures in the proximal femur that developed within 3 months after CT scan, and 1,911 cases of no pathologic fractures in the proximal femur within 3 months after CT scan) to train the fracture prediction artificial neural network model.

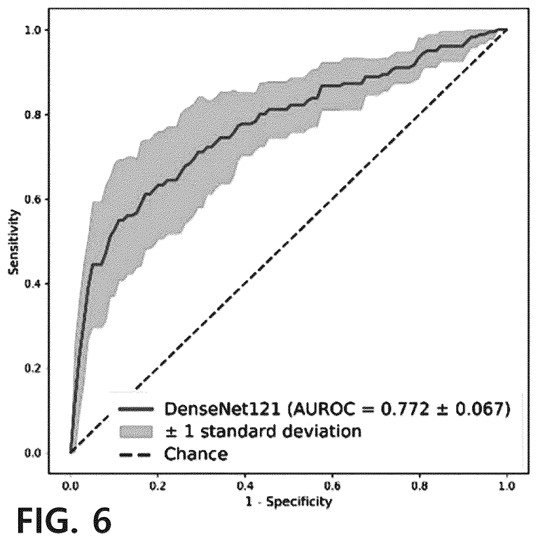

FIG. 6 shows the receiver operating characteristic (ROC) curve when the artificial neural network model used in the device and method for predicting a pathologic fracture in the proximal femur according to an exemplary embodiment of the present invention is configured by DenseNet121, and the input image generated from the CT image is used.

Referring to FIG. 6, the fracture prediction artificial neural network model trained in this way had an area under the curve (AUC) of the receiver operating characteristic (ROC) curve of 0.77±0.07 (0.75 to 0.79) (95% confidence interval).

In addition, as a result of comparing the prediction results of the fracture prediction artificial neural network model trained in this way with the clinician's prediction results for the development of pathologic fractures for the same data, it was confirmed that the prediction results of the trained fracture prediction artificial neural network model were more excellent in terms of specificity and precision.

Specifically, whereas the specificity of the prediction results through the artificial neural network model was 0.98±0.01 (0.98 to 0.99) (95% confidence interval), the specificity of the clinician's prediction results was 0.86±0.09 (0.81 to 0.91) (95% confidence interval). In addition, whereas the precision of the prediction results through the artificial neural network model was 0.72±0.19 (0.67 to 0.77) (95% confidence interval), the precision of the clinician's prediction results was 0.11±0.10 (0.05 to 0.17) (95% confidence interval).

In this way, according to the present invention, an impending pathologic fracture that is difficult for a clinician to predict can be accurately predicted through an abdominal radiograph in which a pelvic CT image is digitally reconstructed. Therefore, it can be clinically helpful to medical oncologists, radiation oncologists and orthopaedic oncologists.

Although the exemplary embodiments of the present invention have been described above, the spirit of the present invention is not limited to the exemplary embodiments presented in the present specification, and those skilled in the art who understand the spirit of the present invention may easily suggest other exemplary embodiments by changing, modifying, deleting or adding components within the scope of the same spirit, but this will also fall within the scope of the present invention.

### REFERENCE NUMERALS

100: Device for predicting pathologic fracture in proximal femur
110: Memory
120: Processor

1. A device for predicting a pathologic fracture in the proximal femur of an advanced cancer patient, comprising:
a memory for storing one or more instructions; and
a processor for executing the one or more instructions,
wherein the processor processes a CT image including the femur of the cancer patient to generate an input image by executing the one or more instructions, inputs the input image into a fracture prediction artificial neural network model, and obtains prediction information about whether a pathologic fracture in the proximal femur will occur within a predetermined period from the time of acquiring the CT image which is output by the fracture prediction artificial neural network model.

2. The device of claim 1, wherein the CT image is a CT image of the abdomen and pelvis of the cancer patient.

3. The device of claim 1, wherein the processor reconstructs the CT image into a radiograph image to generate the input image.

4. The device of claim 3, wherein the CT image is a three-dimensional image and the radiograph image is a two-dimensional image, and
wherein the processor converts the CT image into the radiograph image through perspective projection.

5. The device of claim 3, wherein the processor generates a cropped radiograph image by extracting a part representing the femur of the cancer patient from the reconstructed radiograph image.

6. The device of claim 5, wherein the processor augments the cropped radiograph image to generate a plurality of input images from one of the cropped radiograph images.

7. The device of claim 1, wherein the fracture prediction artificial neural network model is trained by receiving inputs of a learning input image generated by processing abdomen and pelvis CT images of a plurality of cancer patients, and the determination of whether a pathologic fracture in the proximal femur will occur in each of the plurality of cancer patients within a predetermined period from the time of acquiring the CT images, and
wherein the learning input image is generated by respectively reconstructing the abdomen and pelvis CT images of the plurality of cancer patients into radiograph images, and extracting a part representing the femur of each of the cancer patients from each of the reconstructed radiograph images such that a generated femur cropped image is augmented.

8. A method for predicting a pathologic fracture in the proximal femur of an advanced cancer patient, comprising the steps of:
receiving an input of a CT image including the femur of the cancer patient;
generating an input image by processing the CT image; and
inputting the input image into a fracture prediction artificial neural network model, and obtaining prediction information about whether a pathologic fracture in the proximal femur will occur within a predetermined period from the time of acquiring the CT image which is output by the fracture prediction artificial neural network model.

9. The method of claim 8, wherein the CT image is a three-dimensional CT image of the abdomen and pelvis of the cancer patient.

10. The method of claim 9, wherein the step of generating an input image comprises the steps of:
reconstructing the three-dimensional CT image into a two-dimensional radiograph image;
generating a cropped radiograph image by extracting a part representing the femur of the cancer patient from the reconstructed radiological image; and
augmenting the cropped radiograph image to generate a plurality of input images from one of the cropped radiograph images.

11. The method of claim 10, wherein the three-dimensional CT image is reconstructed into the two-dimensional radiograph image through perspective projection.

12. The method of claim 8, wherein the fracture prediction artificial neural network model is trained by receiving inputs of a learning input image generated by processing abdomen and pelvis CT images of a plurality of cancer patients, and the determination of whether a pathologic fracture in the proximal femur will occur in each of the plurality of cancer patients within a predetermined period from the time of acquiring the CT images, and
wherein the learning input image is generated by respectively reconstructing the abdomen and pelvis CT images of the plurality of cancer patients into radiograph images, and extracting a part representing the femur of each of the cancer patients from each of the reconstructed radiograph images such that a generated femur cropped image is augmented.

13. A non-transitory computer-readable storage medium storing a program including one or more instructions for performing the method according to claim 8.
